(19) United States
(12) Patent Application Publication
(10) Pub. No.: US 2015/0037361 A1
(43) Pub. Date: Feb. 5, 2015

(54) COMPOSITIONS AND METHODS TO TREAT THE BIHORMONAL DISORDER IN DIABETES

(71) Applicants: ROGER UNGER, DALLAS, TX (US); JEF DE BRABANDER, Flower Mound, TX (US); MICHAEL ROTH, DALLAS, TX (US); MATTHEW EVANS, Fort Worth, TX (US)

(72) Inventors: ROGER UNGER, DALLAS, TX (US); JEF DE BRABANDER, Flower Mound, TX (US); MICHAEL ROTH, DALLAS, TX (US); MATTHEW EVANS, Fort Worth, TX (US)

(21) Appl. No.: 14/448,247

(22) Filed: Jul. 31, 2014

Related U.S. Application Data
(60) Provisional application No. 61/861,784, filed on Aug. 2, 2013.

(51) Int. Cl. A61K 39/00

(52) U.S. Cl. CPC A61K 39/00
USPC 424/184.1

(57) ABSTRACT

The described invention provides methods and compositions for treating diabetes and hyperglycemia in a mammal comprising administering to the mammal a therapeutic amount of a glucagon depleting compound. Also provided are methods and compositions for eliciting a bihormonal response in a mammal comprising administering to the mammal a therapeutic amount of a glucagon depleting compound. The glucagon depleting compound is effective to normalize glucagon, insulin, glucose, glycated hemoglobin (HgAlC) and C peptide in a mammal suffering from diabetes.

### CROSS-REFERENCE TO RELATED APPLICATIONS

This application claims the benefit of U.S. Provisional Application No. 61/861,784, filed on Aug. 2, 2013, which is hereby incorporated by reference in its entirety.

### FIELD OF THE INVENTION

The described invention relates to glucose homeostasis, the hormones involved therein, diabetes, and therapeutic agents and methods to treat the bihormonal disorder in diabetes.

### BACKGROUND OF THE INVENTION

### Glucose Homeostasis

Glucose, a fundamental source of cellular energy, is released by the breakdown of endogenous glycogen stores that are primarily located in the liver. Glucose is also released indirectly in the muscle through intermediary metabolites. These whole-body energy stores are replenished from dietary glucose, which, after being digested and absorbed across the gut wall, is distributed among the various tissues of the body. (Bryant, N J et al, Nat. Rev. Mol. Cell. Biol. 2002: 3(4): 267-77).

Normally, following glucose ingestion, the increase in plasma glucose concentration triggers insulin release, which stimulates splanchnic (liver and gastrointestinal tissue) and peripheral glucose uptake and suppresses endogenous (primarily hepatic) glucose production. In healthy adults, blood glucose levels are tightly regulated within a range of 70 to 99 mg/dL, and maintained by specific hormones (e.g., insulin, glucagon, incretins) as well as the central and peripheral nervous system, to meet metabolic requirements. Various cells and tissues within the brain, muscle, gastrointestinal tract, liver, kidney and adipose tissue also are involved in blood glucose regulation by means of uptake, metabolism, storage and secretion. (DeFronzo, R A., Med. Clin. N. Am. 88: 787-835 (2004); Gerich, J E, Diabetes Obes. Metab. 2000:2:345-350). Under normal physiologic circumstances, glucose levels rarely rise beyond 140 mg/dL, even after consumption of a high-carbohydrate meal.

Insulin, a potent antilipolytic (inhibiting fat breakdown) hormone, is known to reduce blood glucose levels by accelerating transport of glucose into insulin-sensitive cells and facilititating its conversion to storage compounds via glycogenesis (conversion of glucose toglycogen) and lipogenesis (fat formation); within the islets of Langerhans of the pancreas, 13 cells produce insulin.

Glucagon, a hormone that also plays a role in glucose homeostasis, is produced by a cells within the islets in response to low normal glucose levels or hypoglycemia, and acts to increase glucose levels by accelerating glycogenolysis and promoting gluconeogenesis. After a glucose-containing meal, glucagon secretion is inhibited by hyperinsulinemia, which contributes to suppression of hepatic glucose production and maintenance of normal postprandial glucose tolerance.

Incretins, which include glucose-dependent insulinotropic polypeptide (GIP) and glucagon-like peptide 1 (GLP-1), are also involved in regulation of blood glucose, in part by their effects on insulin and glucagon. (Drucker, and Nauk, Lancet 368: 1696-1705 (2006). Both GLP-1 and GIP are considered glucose-dependent hormones, meaning they are secreted only when glucose levels rise above normal fasting plasma glucose levels. Normally, these hormones are released in response to meals and, by activating certain receptors on pancreatic β cells, they aid in stimulation of insulin secretion. When glucose levels are low, however, GLP-1 and GIP levels (and their stimulating effects on insulin secretion) are diminished. (Drucker, Cell Metab. 3: 153-165 (2006)).

The preproglucagon-derived peptides glucagon, GLP1 and GLP2, are encoded by the preproglucagon gene, which is expressed in the central nervous system, intestinal L-cells, and pancreatic and gastric α cells. A post-translational cleavage by prohormone convertases (PC) is responsible for the maturation of the preglucagon hormone that generates all these peptides. The expression of different PC subtypes in each tissue mediates the production of each different peptide. In a cells, the predominance of proprotein convertase subtilisin/kexin type 2 (PCSK2) leads to production of glucagon together with the products glicentin, glicentin-repeated pancreatic polypeptide, intervening peptide 1 and the major proglucagon fragment (Dey A et al., Endocrinol. 146: 713-727 (2005)). In enteroendocrine cells, PCSK1/3 enzymes cleave the preproglucagon hormone to generate GLP1 and GLP2 along with glicentin, intervening peptide 1 and oxyntomodulin (Mojsov S, J. Biol. Chem. 261: 11880-11889 (1986)). Under certain conditions, islet α cells are an extraintestinal site for GLP-1 production. (Portha B, et al. Exptl Diabetes Res. 2011, Article 376509). One of the many observed cellular effects of GLP-1 is the inhibition of β cell K_{ATP} channels, which initiates Ca2+ influx through voltage-dependent calcium channels and triggers the exocytotic release of insulin (MacDonald, P E et al., Diabetes 51 (Suppl. 3): S434-S442 (2002).

Transport of Glucose into Cells

Since glucose cannot readily diffuse through all cell membranes, it requires assistance from both insulin and a family of transport proteins (facilitated glucose transporter [GLUT] molecules) in order to gain entry into most cells. (Bryant, et al, Nat. Rev. Mol. Cell. Biol. 2002; 3(4): 267-277). GLUTs act as shuttles, forming an aqueous pore across otherwise hydrophobic cellular membranes, through which glucose can move more easily. Of the 12 known GLUT molecules, GLUT4 is considered the major transporter for adipose, muscle, and cardiac tissue, whereas GLUTs 1, 2, 3, and 8 facilitate glucose entry into other organs (eg, brain, liver). Activation of GLUT4 and, in turn, facilitated glucose diffusion into muscle and adipose tissue, is dependent on the presence of insulin, whereas the function of other GLUTs is more independent of insulin. (Uldry, M, Thorens B, Eur. J. Physiol. 2004; 447: 480-489).

The majority of glucose uptake (≧80%) in peripheral tissue occurs in muscle, where glucose may either be used immediately for energy or stored as glycogen. Skeletal muscle is insulin-dependent, and thus requires insulin for activation of glycogen synthase, the major enzyme that regulates production of glycogen. While adipose tissue is responsible for a much smaller amount of peripheral glucose uptake (2%-5%), it plays an important role in the maintenance of total body glucose homeostasis by regulating the release of free fatty acids (which increase gluconeogenesis) from stored triglycerides, influencing insulin sensitivity in the muscle and liver.

While the liver does not require insulin to facilitate glucose uptake, it does need insulin to regulate glucose output. Thus, for example, when insulin concentrations are low, hepatic glucose output rises. Additionally, insulin helps the liver store most of the absorbed glucose in the form of glycogen.

The kidneys play a role in glucose homeostasis via release of glucose into the circulation (gluconeogenesis), uptake of glucose from the circulation to meet renal energy needs, and reabsorption of glucose at the proximal tubule. The kidneys also aid in elimination of excess glucose (when levels exceed approximately 180 mg/dL, though this threshold may rise during chronic hyperglycemia) by facilitating its excretion in the urine. In diabetes mellitus where glucose levels are high and may exceed the threshold of glucose reabsorption, more glucose may be excreted in the urine if concentrations in filtered urine become high.

Cytoarchitecture of Human Islets

There is a wealth of information about the physiology of rodent islets of Langerhans, but the biology of human islets remains poorly understood. The islets of Langerhans are small organs located in the pancreas that are crucial for glucose homeostasis. Islets typically consist of four types of secretory endocrine cells, namely, the insulin-containing β cells, the glucagon-containing a cells, the somatostatin-containing δ cells, and the pancreatic polypeptide-producing (PP) cells. Human islets are composed of about 60% β cells and about 30% α cells (8-10, 15), with somatostatin- and PP-expressing cells constituting a minority in islets. Somatostatin works as an inhibitor of both glucagon and insulin release. (Fehmann, H C, et al, Am. J. Physiol, Endocrino & Metab. 268: E40-47 (1995)). Immunocytochemical studies in human islets have shown that among the five identified somatostatin receptor (SSTR) subtypes, SSTR2 is highly expressed in α cells, while SSTR1 and SSTR5 are expressed in β cells. (Kumar U et al., Diabetes 48: 77-85 (1999)).

In rodent islets, the vastly predominating β cells are clustered in the core of a generally round islet, surrounded by a mantle of α, δ, and PP cells. In contrast, in human islets, insulin-containing β cells intermingle with other cell types within the islet, i.e., insulin-, glucagon-, and somatostatin-containing cells are found distributed throughout the human islet (Cabrera, et al., Proc. Natl. Acad. Sci. U.S. 103: 2334-2339 (2006)). Human islets do not show obvious subdivisions, but 90% of α-cells are in direct contact with β cells, and β cells intermingled freely with other endocrine cells throughout the islet. β, α, and δ cells had equivalent and random access to blood vessels within the islet, ruling out the possibility that the different endocrine cells are organized in layers around blood vessels. These results support a model in which there is no set order of islet perfusion and in which any given cell type can influence other cell types, including its own cell type (G. da Silva Xavier et al., Diabetologia 54, 819 (2011)).

Thus, in human islets, the α and β cells are mixed together, with more than 70% of β cells in contact with non-β cells. Although this arrangement predisposes human islets for strong paracrine interactions, how human islet architecture affects cell-to-cell interactions that lead to regulated and concerted hormonal secretion remains to be determined. Given their large contribution to the islet and their close association with β cells, α cells may exert a stronger influence on the overall activity of the human islet than in rodent islets.

In normal humans and rodents, β cells respond to glucose with an immediate short-lived spike of insulin release, followed early in its downward course by a prolonged second phase. (Unger and Orci, Proc. Natl. Acad. Sci., U.S., 107 (37): 16009-16012 (2010)). Normally, the insulin spike is accompanied by a reciprocal decrease in glucagon, consistent with the possibility that glucagon exerts a paracrine suppressive effect on a cells. Conversely, when insulin levels decrease in response to a decrease in glucose, normally a reciprocal increase in glucagon occurs. It has been proposed that these reciprocal changes in insulin and glucagon provide the defense against glycemic perturbations that is characteristic of normal human glucose homeostasis.

Normal regulation of a cells requires the juxtaposed presence of normally functioning β cells. According to the Sherringtonian model of coordinated reciprocal paracrine hormone secretion in the regulation of homeostasis of fuel production and utilization, the β cell controls basal a cell secretion of glucagon via tonic paracrine inhibition. When a carbohydrate-containing meal stimulates insulin secretion, the paracrine insulin promptly suppresses the α cells to reduce glucagon secretion, which not only lowers hepatic fuel production, but permits it to metabolize incoming glucose to glycogen. Meanwhile, the endocrine insulin that enters the systemic circulation reduces hepatic fuel production by opposing glucagon action and also enhancing glucose uptake by skeletal muscle and adipocytes (Unger and Orci, Proc. Natl. Acad. Sci., U.S., 107 (37): 16009-16012 (2010)).

Glucagon Control of Glucose Homeostasis and Metabolism

Glucagon plays a central role in the response to hypoglycemia and also opposes insulin effects. The main action of glucagon occurs in the liver, where the insulin/glucagon ratio controls multiple steps of hepatic metabolism. Glucagon stimulates gluconeogenesis and glycogenolysis, which increases hepatic glucose output, ensuring an appropriate supply of glucose to body and brain, and at the same time, it decreases glycogenesis and glycolysis. The glycogen receptor in the liver is highly selective for glucagon, but it exhibits a modest affinity for glucagon-like peptides (Hjorth, S A, et al, J. Biol. Che. 269: 30121-30124 (1994)). Its main action on the liver is mediated by the activation of adenylyl cyclase and the protein kinase A (PKA) pathway.

As shown in FIG. 1 (Quesada, I. et al., J. Endocrinol. 199: 5-19 (2008)), glucagon regulates gluconeogenesis mainly by the up-regulation of key enzymes, such as glucose-6-phosphatase (G6PC) and phosophoenolpyruvate carboxykinase (PCK2) through the activation of the cAMP response element binding protein (CREB) and peroxisome proliferator-activated receptor γ-coactivator-1 (PPARGC1A). (Herzig, S., et al, Nature 413, 179-183 (2001); Yoon, J C et al, Nature 413: 131-138 (2001)). PCK2 and G6PC along with fructose-1.-biphosphatase (FBP1) play a key role in the rate of gluconeogenesis. PCK2 mediates the conversion of oxaloacetate into phosphoenolpyruvate, while G6PC regulates glucose production from glucose-6-phoshate. FBP1 converts fructose-1,6-biphosphate (F(1,6)P2) into fructose-6-phosphate (F6P). Its activity is regulated by glucagon: it decreases the intracellular levels of fructose-1,6-biphosphate (F(2,6)P2), an allosteric inhibitor of FBP1 (Kurland I J and Pilkin, S J, Protein Sci. 4: 1023-37 (1995)). This decrease in F(2,6)P2 also reduces the activity of phosphofructokinase-1 (PFKM), downregulating glycolysis. The glycolytic pathway is further inhibited by glucagon at the pyruvate kinase (PKLR) level (Slavin, B G et al, J. Lipid Res. 35: 1535-1541 (1994)). Glycogen metabolism is primarily determined by the activity of glycogen synthase (GS) and glycogen phosphorylase (GP). While glucagon is important for GP phosphorylation and activation, it inhibits GS function by inducing its phosphorylation and its conversion into an inactive form (Band G C & Jones C T, FEBS Letters 119: 190-194 (1980), Ciudad, C. et al, Eur. J. Biochem. 142: 511-520 (1984); Andersen B et al, Biochem. J. 342: 545-550 (1999).

Glucagon also stimulates the uptake of amino acids for gluconeogenesis in the liver, and is involved in the regulation of fatty acids in adipocytes. An elevated glucagon to insulin ratio accelerates gluconeogenesis as well as fatty acid β oxidation and ketone body formation (Vons, C et al, Hepatology 13: 1126-1130 (1991).

The Pancreatic α Cell and Glucagon Secretion

In isolated rat and mouse α cells, in addition to the effects of insulin and somatostatin on α cells, glucagon itself works as an extracellular messenger by exerting an autocrine positive feedback that stimulates secretion by an increase in exocytosis associated with a rise in cAMP levels. (Ma X et al, Molec. Endocrinol. 19: 198-212 (2005).

In a normal subject, whenever there is an increased demand for glucose (e.g., starvation, hypoglycemia, and exercise), insulin secretion falls, stimulating glucagon secretion. This removes insulin's inhibitory action on the liver while augmenting glucagon's stimulatory effect on fuel production. As a result, glucose production is increased to meet the needs of the organism. When glucose is abundant, as with an oral glucose load, the reverse occurs. See, Unger R H and A D Cherrington, J. Clin. Invest. 122(1): 4-12 (2012).

Accordingly, both insulin and glucagon are needed in physiologic balance for glucose homeostasis, and the relationship between α- and β-cells in juxtaposition in human islets is crucial to maintain that balance.

### Diabetes Mellitus

Diabetes mellitus is a group of metabolic diseases characterized by hyperglycemia. Chronic hyperglycemia is associated with long-term damage, dysfunction, and potential failure of organs, including the eyes, kidneys, nerves, heart and blood vessels. It is best appreciated as a bihormonal disorder. (See, e.g., Unger R H and A D Cherrington, J. Clin. Invest. 122(1): 4-12 (2012)).

### Type 1 Diabetes Mellitus (T1DM)

In type 1 diabetes mellitus, β cells are destroyed by an autoimmune process and largely replaced by α cells. (Unger and Orci, Proc. Natl. Acad. Sci., U.S., 107 (37): 16009-16012 (2010)). These α cells lack the tonic restraint normally provided by the high local concentrations of insulin from juxtaposed β cells, resulting in inappropriate hyperglucagonemia. (P. Raskin, R. H. Unger, Glucagon and diabetes. The Medical Clinics of North America 62, 713 (1978); J. F. Habener et al., Trends in Endocrinology & Metabolism: TEM 24, 153 (2013); R. H. Unger and A. D. Cherrington, J. Clinical Investig. 122, 4 (2012); P. M. Vuguin and M. J. Charron, Diabetes, Obesity & Metabolism 13 Suppl 1, 144 (2011)), which drives surges of hyperglycemia which increases glucagon secretion (See Unger R H and A D Cherrington, J. Clin. Invest. 122(1): 4-12 (2012). Supernormal insulin levels are needed to match the insulin that neighboring β cells give to a cells in normal islets. This results in lifelong hyperinsulinemia, which exposes the subject to frequent incidences of hypoglycemia, which increases such sequelae as accumulation of low density lipoprotein (LDL) in the walls of blood blood vessels, causing the blockages of atherosclerosis, and coronary artery disease.

The lethal catabolic consequences of complete insulin deficiency do not occur if the hyperglucagonemia is suppressed by somatostatin (Raskin & Unger (NEJM 299: 433-436 (1978); Unger R H and A D Cherrington, J. Clin. Invest. 122(1): 4-12 (2012), showing in FIG. 4 that somatostatin eliminates glucose spikes in streptozotocin treated mice)), by the hormone leptin (Wang M, et al, Proc. Natl. Acad. Sci., U.S., 107: 4813-4819 (2010); Yu et al, Proc. Natl. Acad. Sci., U.S., 105: 14070-14075 (2008), if its action is blocked (e.g., by glucagon receptor antagonists (Rivera, N. et al, J. Pharmcol. Exp. Thera. 321: 743-752 (2007), or by genetic disruption of glucagon receptors (Lee, Y, et al, Diabetes 60: 391-397 (2011).

Although normalizing high glucose levels suppress glucagon and insulin spikes while satisfying the glucose requirements of muscles, insulin replacement alone does not correct the defect in glucose metabolism because there are surges of glucagon during hyperglycemia incidences (Taki K et al., Diabetes Technol. & Therapeutics, July 2010, 12 (7): 523-528; Derr, E. et al. Diabetes Care 26: 2728-2733 (2003)).

The ideal therapeutic agent for diabetes has yet to be developed. The overlap in human islets between the different SSTR subtypes in α and β cells limit the use of subtype-specific somatostatin analogs (Singh, V et al., J. Clin. Endocrinol & Metab. 92: 673-680 (2007)), and while leptin suppresses glucagon, and maintains normal glycemia, its pleiotropic effects limit its use.

Several linear and cyclic peptide-based glucagon analogues have been developed as glucagon receptor antagonists; for example, des-His¹, des-Phe⁶, Glu⁹] glucagon-NH₂ reduces glucose levels in steptozotocin-induced diabetic rats (Van Tine, B A et al., Endocrinol. 137: 3316-3322 (1996)), and the antagonist des-His glucagon binds preferentially to the hepatic glucagon receptor in vivo, which correlates with its glucose lowering effects (Dallas-Yang, Q et al., Eur. J. Pharmcol. 501: 225-234 (2004)).

Multiple competitive and non-competitive, nonpeptide antagonists have been reported to act on glucagon binding and/or function, although the information about the effect of these agonists on humans is scarce. Bay 27-9955, an oral glucagon receptor antagonist that has been tested in humans, reduces glucose levels induced by exogenous glucagon (Petersen, K F & Sullivan J T, Diabetologia 44: 2018-2024 (2001)).

Studies using knock-out mice have shown that glucagon is essential in the pathogenesis of diabetes. For example, Lee et al. (Diabetes, Vol. 60, No. 2, pp. 391-397 (2011)) destroyed β-cells in 10- to 12-week-old GcgR^{-/-} and GcgR^{+/+} (WT) mice with two intravenous injections of streptozotocin (STZ) (100 mg/kg body weight followed in 7 days by 80 mg/kg) in order to completely eliminate the source of insulin in these mice (Lee et al., Diabetes, Vol. 60, No. 2, pp. 391-397 (2011)). To assess the completeness of β-cell destruction, each mouse was sacrificed after experimentation and the pancreas was processed for morphometric quantification of immunocytochemically positive insulin containing cells (Lee et al., Diabetes, Vol. 60, No. 2, pp. 391-397 (2011)). STZ-induced β-cell destruction caused severe hyperglycemia in WT mice while a similar degree of β-cell destruction in GcgR^{-/-} mice did not alter glucose levels (Lee et al., Diabetes, Vol. 60, No. 2, pp. 391-397 (2011)). Indeed, STZ-treated GcgR^{-/-} mice appeared to be in a seemingly normal state of health (Lee et al., Diabetes, Vol. 60, No. 2, pp. 391-397 (2011)). Lee et al. concluded that the metabolic manifestations of insulin deficiency cannot occur in the absence of glucagon (Lee et al., Diabetes, Vol. 60, No. 2, pp. 391-397 (2011)).

However, whether the glucagon receptor is antagonized or knocked out, there is compensation by the glucagon gene, which regulates glucagon and GLP-1, to try to overcome the glucagon blockade. High levels of insulin therefore are maintained when glucagon is suppressed, altering the insulin-glucagon opposition. Thus, antagonizing the glucagon receptor action results in a double pathology—the original lack of insulin plus overproduction of glucagon in response to receptor antagonism (W. Gu et al., Endocrinology & Metabolism 299, E624 (2010) and N. Rivera et al., J. Pharmacol. & Exptl Therapeutics 321, 743 (2007)), and the hypergluconemia that results from receptor blockage requires almost continual occupancy of the receptor to prevent glucagon signaling, which will require high blood concentrations of receptor antagonists.

Other available agents also are not without undesirable side effects. For example, in humans, sulphonylureas (such as glibenclamide, tolbutamide) are associated with a decrease in glucagon secretion in healthy and type 2 diabetic subjects (Landstedt-Hallin L et al., J. Clin. Endocrinol. & Metab. 84: 3140-3145 (1999)), while they stimulate glucagon levels in type 1 diabetic subjects Bohannon N V et al, J. Clinical Endocrinol. & Metab. 54: 459-462 (1982)). They also induce insulin and somatostatin secretion.

In addition to stimulating insulin release, GLP1 can suppress glucagon secretion in humans in a glucose-dependent manner. GLP1 is rapidly cleaved and inactivated by the enzyme dipeptidyl peptidase-IV (DPP4). Examples of GLP1 inhibitors include exenatide (Byetta®, Bydureon®), a synthetic polypeptide with high resistance to DPP4 cleavage and liraglutide (Victoza®). DPP4 inhibitors, for example, sitagliptin (Januvia®), vildagliptin, saxagliptin (Onglyza®), and linagliptin (Trajenta®), increase the endogenous effects of GLP1, reducing glucagon plasma concentrations in diabetic subjects (Rosenstock J et al., Diabetes Care 30: 217-223 (2007). All produce opposing actions on insulin and glucagon. However, DPP4 processes 20 hormones, which accounts for its side effects.

A more physiologic way to treat T1D would be to restore normal physiologic glucose levels by depleting glucagon and normalizing insulin.

The described invention provides synthetic compounds that specifically suppress glucagon mRNA synthesis in the alpha cell. Depleting the stores of glucagon allows greater latitude for the fluctuating levels of the inhibitor that occur with oral administration, because it takes hours to restore glucagon stores after the inhibitor falls below its effective concentration. These orally available small molecules normalize four pathological characteristics of T1D: blood glucose levels, hemoglobin AlC, glucagon and C peptide. While excess glucagon is depleted, thereby eliminating hyperglycemic surges that now complicate the management of type 1 diabetes. Since well-controlled insulin-treated patients with type 1 diabetes are hyperinsulinemic (Wang M Y et al, J. Diabetes Complications 2013 January-February; 27(1): 7074) and their glucagon response to hypoglycemia is therefore absent, glucagon depletion therapy imposes no additional hypoglycemic risk. In fact, since insulin dosage is drastically reduced, bihormonal therapy can eliminate hypoglycemia.

### SUMMARY OF THE INVENTION

The present disclosure provides methods and compositions useful in treating diabetes.

According to one aspect, the described invention provides a method for treating diabetes in a mammal comprising administering to the mammal a therapeutic amount of a glucagon depleting compound, wherein the therapeutic amount of the glucagon depleting compound is effective to elicit a bihormonal response in the mammal.

According to another aspect, the described invention provides a method for treating hyperglycemia in a mammal comprising administering to the mammal a therapeutic amount of a glucagon depleting compound, wherein the therapeutic amount of the glucagon depleting compound is effective to elicit a bihormonal response in the mammal.

According to another aspect, the described invention provides a method for eliciting a bihormonal response in a mammal suffering from diabetes comprising administering to the mammal a therapeutic amount of a glucagon depleting compound.

According to one embodiment, the bihormonal response is a normalization of glucagon levels and a normalization of insulin levels.

According to one embodiment, the therapeutic amount of the glucagon depleting compound is effective to normalize blood glucose levels in the mammal. According to another embodiment, the therapeutic amount of the glucagon depleting compound is effective to normalize glycated hemoglobin (HgAlC) levels and advanced glycation end-products in the mammal. According to another embodiment, the therapeutic amount of the glucagon depleting compound is effective to normalize glucagon levels in the mammal. According to another embodiment, the therapeutic amount of the glucagon depleting compound is effective to normalize C peptide levels in the mammal. According to another embodiment, the therapeutic amount of the glucagon depleting compound is effective to inhibit the expression of glucagon mRNA.

According to one embodiment, the diabetes is Type 1 diabetes. According to another embodiment, the diabetes is Type 2 diabetes.

According to one embodiment, the hyperglycemia is associated with Type 1 diabetes. According to another embodiment, the hyperglycemia is associated with Type 2 diabetes.

### BRIEF DESCRIPTION OF THE DRAWINGS

FIG. 1 shows a schematic representation of glucagon receptor signaling.

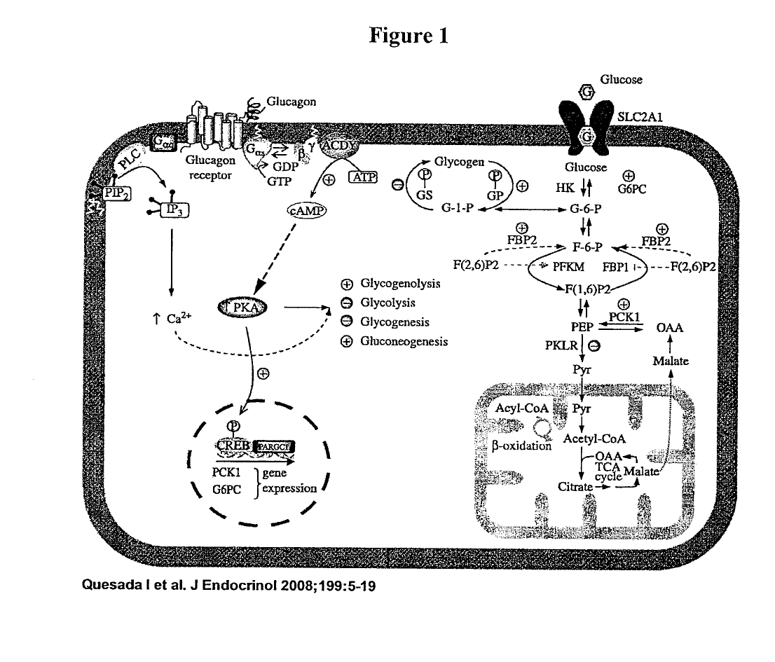

FIG. 2 shows a dose response curve measuring the inhibitory effect of glucagon depleting Compound A on glucagon mRNA expression in InR1G9 cells.

FIG. 3 shows blood glucose levels measured in four non-obese diabetic (NOD) mice, an animal model for T1D, treated with saline (squares) and four NOD mice treated with Compound A (circles).

FIG. 4 shows triglyceride levels measured in a wild-type (wt) mouse, in an NOD mouse treated with saline and in an NOD mouse treated with Compound A.

FIG. 5 shows blood glucose levels, hemoglobin AlC levels, glucagon levels and C-peptide levels measured in NOD mice treated with saline, NOD mice treated with Compound A and new onset diabetic mice. Levels were normalized to wild-type (non-diabetic) mice.

FIG. 6 shows blood glucose levels in diet-induced diabetic mice treated with Compound A.

### DETAILED DESCRIPTION OF THE INVENTION

The described invention can be better understood from the following description of exemplary embodiments, taken in conjunction with the accompanying figures and drawings. It should be apparent to those skilled in the art that the described embodiments of the described invention provided herein are merely exemplary and illustrative and not limiting.

The terms “AlC, glycated hemoglobin, glycosylated hemoglobin, hemoglobin AlC, HgAlc, and HbAlc” are used interchangeably herein to describe a diagnostic test that measures the percentage of hemoglobin coated with sugar (glycated), which reflects average blood glucose for the past 2 to 3 months. The higher the AlC level, the poorer the blood sugar control and the higher the risk of diabetes complications. Diabetes is diagnosed at an AlC of ≧6.5%.

The term “administer”, “administering” or “to administer” as used herein, refers to the giving or supplying of a medication, including in vivo administration, as well as administration directly to tissue ex vivo. Generally, compositions may be administered systemically either orally, bucally, parenterally, topically, by inhalation or insufflation (i.e., through the mouth or through the nose) or rectally in dosage unit formulations containing conventional nontoxic pharmaceutically acceptable carriers, adjuvants and vehicles as desired, or may be locally administered by means such as, but not limited to, injection, implantation, grafting, topical application or parenterally.

The terms “agent” and “therapeutic agent” are used interchangeably herein to refer to a drug, molecule, composition, or other substance that provides a therapeutic effect. The term “active agent” as used herein, refers to the ingredient, component or constituent of the compositions of the described invention responsible for the intended therapeutic effect.

The term “alpha cell” or “α-cell” as used herein refers to a type of cell in the pancreas. Alpha cells make and release a hormone called glucagon when the blood glucose level falls too low. Glucagon stimulates the liver to release glucose into the blood for energy.

The term “amylin” as used herein refers to a hormone formed by beta cells in the pancreas. Amylin regulates the timing of glucose release into the bloodstream after eating by slowing the emptying of the stomach.

The term “atherosclerosis” as used herein refers to a clogging, narrowing and hardening of the body's large arteries and medium-sized blood vessels. Atherosclerosis can lead to stroke, heart attack, eye problems and kidney problems.

The term “autocrine signaling” refers to a type of cell signaling in which a cell secretes signal molecules that act on itself or on other adjacent cells of the same type.

The term “autoimmune disease” as used herein refers to a disorder of the body's immune system in which the immune system mistakenly attacks and destroys body tissue that it believes to be foreign.

The term “autonomic neuropathy” as used herein refers to a type of neuropathy affecting the lungs, heart, stomach, intestines, bladder or genitals.

The terms “background retinopathy”, “simple retinopathy”, and “nonproliferative retinopathy” are used interchangeably to refer to degenerative changes in retinal capillaries, which are an early stage of diabetic retinopathy.

The term “Beta cells” or “β-cells” as used herein refers to a pancreatic cell that makes insulin.

The terms “blood glucose” or “blood sugar” are used interchangeably to refer to the main sugar found in the blood and the body's main source of energy.

The term “blood glucose level” as used herein refers to the amount of glucose in a given amount of blood. It is noted in milligrams in a deciliter, or mg/dL.

The term “blood glucose meter” as used herein refers to a small, portable machine used by those with diabetes to check blood glucose levels. After pricking the skin with a lancet, a drop of blood is placed on a test strip in the machine. The meter (or monitor) displays the blood glucose level as a number on the meter's digital display.

The term “blood glucose monitoring” as used herein refers to checking blood glucose level on a regular basis in order to manage diabetes.

The term “C peptide (or connecting peptide)” as used herein refers to a substance released by the pancreas into the bloodstream in equal amounts to insulin. A test that measures the level of C-peptide reflects how much insulin the body is making

The term “cardiovascular disease” as used herein refers to a disease of the heart and blood vessels (arteries, veins and capillaries).

The term “cerebrovascular disease” as used herein refers to a disease of the brain and its blood vessels (arteries, veins and capillaries).

The term “complications” as used herein refers to harmful effects of diabetes such as damage to the eyes, heart, blood vessels, nervous system, teeth and gums, feet and skin, or kidneys. Studies show that keeping blood glucose, blood pressure, and low-density lipoprotein cholesterol levels close to normal can help prevent or delay these problems.

The term “condition” as used herein, refers to a variety of health states and is meant to include disorders or diseases caused by injury or any underlying mechanism or disorder.

The term “creatinine” as used herein refers to a waste product from protein in the diet and from the muscles of the body. Creatinine is removed from the body by the kidneys; as kidney disease progresses, the level of creatinine in the blood increases.

The term “derivative” as used herein, refers to a compound that may be produced from another compound of similar structure in one or more steps. A “derivative” or “derivatives” of a compound retain(s) at least a degree of the desired function of the compound. Accordingly, an alternate term for “derivative” may be “functional derivative.” Derivatives can include chemical modifications, such as alkylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

The term “diabetes mellitus” as used herein refers to a chronic metabolic disorder in which utilization of carbohydrate is impaired and that of lipid and protein enhanced. It is caused by an absolute or relative deficiency of insulin and is characterized, if left untreated, by chronic hyperglycemia, glucosuria, water and electrolyte loss, ketoacidosis and coma. Long-term complications include neuropathy, retinopathy, nephropathy, generalized degenerative changes in large and small blood vessels, and increased susceptibility to infection.

The term “diabetic myelopathy” as used herein refers to damage to the spinal cord found in some individuals with diabetes.

The term “diabetic retinopathy” as used herein refers to retinal changes occurring in diabetes mellitus, marked by microaneurysms, exudates, which are deposits of lipid and protein from leaking capillaries, and hemorrhages, and sometimes by neovascularization. The principal form is nonproliferative retinopathy.

The terms “disease” or “disorder” as used herein, refers to an impairment of health or a condition of abnormal functioning.

The term “endocrine signaling” as used herein refers to cell to cell communication, whereby an endocrine cell secretes a hormone into the blood that can affect a target cell that may be distant from the signaling cell.

The term “fasting blood glucose test” as used herein refers to a check of a person's blood glucose level after the person has not eaten for 8 to 12 hours (usually overnight). to diagnose pre-diabetes and diabetes and to monitor people with diabetes. Diabetes is diagnosed at fasting blood glucose of greater than or equal to 126 mg/dL.

The term “glucagon” as used herein refers to a hormone produced by the alpha cells in the pancreas that raises blood glucose.

The term “hormone” as used herein refers to a chemical produced in one part of the body and released into the blood to trigger or regulate particular functions of the body.

The term “hyperglycemia” as used herein refers to excess blood glucose. Fasting hyperglycemia is blood glucose above a desirable level after a person has fasted for at least 8 hours. Postprandial hyperglycemia is blood glucose above a desirable level 1 to 2 hours after a person has eaten.

The term “hyperinsulinemia” as used herein refers to a condition in which the level of insulin in the blood is higher than normal.

The term “hyperlipidemia” as used herein refers to a higher than normal fat and cholesterol levels in the blood.

The term “hypoglycemia” as used herein refers to a condition that occurs when one's blood glucose is lower than normal, usually less than 70 mg/dL. Signs include hunger, nervousness, shakiness, perspiration, dizziness or light-headedness, sleepiness, and confusion. If left untreated, hypoglycemia may lead to unconsciousness.

The term “impaired fasting glucose (IFG)” as used herein refers to a condition in which a blood glucose test, taken after an 8- to 12-hour fast, shows a level of glucose higher than normal but not high enough for a diagnosis of diabetes. IFG, also called pre-diabetes, is a level of 100 mg/dL to 125 mg/dL. Most individuals with pre-diabetes are at increased risk for developing type 2 diabetes.

The term “impaired glucose tolerance (IGT)” as used herein refers to a condition in which blood glucose levels are higher than normal but are not high enough for a diagnosis of diabetes. IGT, also called pre-diabetes, is a level of 140 mg/dL to 199 mg/dL 2 hours after the start of an oral glucose tolerance test (OGTT). Most individuals with pre-diabetes are at increased risk for developing type 2 diabetes.

The term “implantable insulin pump” as used herein refers to a small pump placed inside the body to deliver insulin in response to remote-control commands from the user. The pump connects to a narrow, flexible plastic tubing that ends with a needle inserted just under the skin. Users set the pump to give a steady trickle or basal amount of insulin continuously throughout the day. Pumps release bolus doses of insulin (several units at a time) at meals and at times when blood glucose is too high, based on programming done by the user.

The term “insulin” as used herein refers to a hormone made by beta cells of the pancreas that helps the body use glucose for energy. When the body cannot make enough insulin, it is taken by injection or through use of an insulin pump.

The term “insulin adjustment” as used herein refers to a change in the amount of insulin a person with diabetes takes based on factors such as meal planning, activity and blood glucose levels.

The term “insulin analog” as used herein refers to a tailored form of insulin in which certain amino acids in the insulin molecule have been modified. The analog acts in the same way as the original insulin, but with some beneficial differences for people with diabetes.

The term “insulin reaction” as used herein refers to a condition when the level of glucose in the blood is too low (at or below 70 mg/dL). It is also known as hypoglycemia.

The term “insulin resistance” as used herein refers to a condition in which the body's cells' normal response to a given amount of insulin is reduced. As a result, higher levels of insulin are needed for insulin to have its proper effects. The pancreas produces more and more insulin until it no longer can produce sufficient insulin for the body's demands. Blood sugar then rises. Insulin resistance is a risk factor for development of type 2 diabetes.

The term “islet cell autoantibodies (ICA)” as used herein refers to proteins found in the blood of those newly diagnosed with Type 1 diabetes and in those who may be developing Type 1 diabetes. The presence of ICA indicates that the body's immune system has been damaging beta cells in the pancreas.

The term “islets” as used herein refers to groups of cells located in the pancreas that make hormones that help the body break down and use food. For example, alpha cells make glucagon and beta cells make insulin. Also called “islets of Langerhans”.

The term “lancet” as used herein refers to a spring-loaded device used to prick the skin with a small needle to obtain a drop of blood for blood glucose monitoring.

The term “latent autoimmune diabetes in adults (LADA)” as used herein refers to a condition in which Type 1 diabetes develops in adults.

The term “low-density lipoprotein cholesterol” (“LDL cholesterol”)” as used herein refers to low density lipoproteins. A “lipoprotein” is a compound containing both lipid and protein. Lipoproteins are constituents of biological membranes and of myelin. Conjugation with protein facilitates transport of lipids, which are hydrophobic, in the aqueous medium of the plasma. Plasma lipids can be separated by such methods as, for example, ultracentrifugation, electrophoresis or immunoelectrophoresis, and are usually classified according to their densities/flotation constants. The principal classes by density are chylomicrons, which transport dietary cholesterol and triglycerides from the intestine to the liver and other tissues; very low density lipoproteins (VLDL), which transport triglycerides from intestine and liver to muscle and adipose tissue; low density lipoproteins (LDL) which transport cholesterol to tissues other than the liver; and high density lipoproteins (HDL), which transport cholesterol to the liver for excretion in bile. The concentrations of certain serum lipoproteins correlate closely with the risk of atherosclerosis. An HDL cholesterol level below 35 mg/dL, an LDL cholesterol level above 160 mg/dL, and a fasting triglyceride level above 250 mg/dL are all independent risk factors for coronary artery disease.

The term “lipid profile” as used herein refers to a blood test that measures total cholesterol, triglycerides, and HDL cholesterol. LDL cholesterol is then calculated from the results.

The term “liver” as used herein refers to an organ in the body that changes food into energy, removes alcohol and poisons from the blood, and makes bile, a substance that breaks down fats and helps rid the body of wastes.

The term “macrovascular disease” as used herein refers to complications of diabetes that affect the large blood vessels, such as those found in the heart and at the base of the brain.

The term “macula” as used herein refers to an oval area of the sensory retina, 3 by 5 mm, temporal to the optic disk corresponding to the posterior pole of the eye; at its center is the central fovea, which contains only retinal cones.

The term “macular edema” as used herein refers to swelling of the macula.

The term “microvascular disease” as used herein refers to complications of diabetes that affect small blood vessels, e.g., capillaries, such as those found in the eyes (retinopathy), nerves (neuropathy), and kidneys (nephropathy).

The term “nephropathy disease of the kidneys” as used herein refers to damage to the kidney's glomeruli, for example due to hyperglycemia and hypertension. When the kidneys are damaged, protein leaks out of the kidneys into the urine. Damaged kidneys can no longer remove waste and extra fluids from the bloodstream.

The term “neuropathy” as used herein refers to a disease of the nervous system. The three major forms in people with diabetes are peripheral neuropathy, autonomic neuropathy, and mononeuropathy. The most common form is peripheral neuropathy, which affects mainly the legs and feet.

The terms “normal”, “normalizing”, and “normalization” refer to a standard, model, median or average of a large group.

The term “oral glucose tolerance test (OGTT)” as used herein refers to a test to diagnose pre-diabetes and diabetes given by a health care professional after an overnight fast. A blood sample is taken, then the patient drinks a high-glucose beverage. Blood samples are taken at intervals for 2 to 3 hours. Test results are compared with a standard and show how the body uses glucose over time. Diabetes is diagnosed when a 2 hour blood glucose is greater than or equal to 200 mg/dL.

The term “particle”, as used herein, refer to extremely small constituents, (e.g., nanoparticles or microparticles) that may contain in whole or in part therapeutic agent(s). The particles may contain therapeutic agent(s) in a core surrounded by a coating. Therapeutic agent(s) also may be dispersed throughout the particles. Therapeutic agent(s) also may be adsorbed into the particles. The particles may be of any order release kinetics, including zero order release, first order release, second order release, delayed release, sustained release, immediate release, etc., and any combination thereof. The particle may include, in addition to therapeutic agent(s), any of those materials routinely used in the art of pharmacy and medicine, including, but not limited to, erodible, nonerodible, biodegradable, or nonbiodegradable material or combinations thereof. The particles may be microcapsules that contain the therapeutic agent(s) in a solution or in a semi-solid state. The particles may be of virtually any shape.

The term “paracrine signaling” as used herein refers to short-range cell-cell communication via secreted signal molecules that act on adjacent cells.

The term “peripheral neuropathy” as used herein refers to nerve damage that affects the feet, legs, or hands. Peripheral neuropathy can cause pain, numbness, or a tingling feeling.

The term “peripheral vascular disease (PVD)” as used herein refers to a disease of the large blood vessels of the arms, legs, and feet. PVD may occur when major blood vessels in these areas are blocked and do not receive enough blood. The signs of PVD are aching pains and slow-healing foot sores.

The term “pre-diabetes” as used herein refers to a condition in which blood glucose levels are higher than normal but are not high enough for a diagnosis of diabetes. People with pre-diabetes are at increased risk for developing Type 2 diabetes and for heart disease and stroke. Results indicating prediabetes include: an AlC of 5.7%-6.4%, Fasting blood glucose of 100-125 mg/dL, and an oral glucose tolerance test (OGTT) 2 hour blood glucose of 140 mg/dL-199 mg/dL.

The term “prevent” in its various grammatical forms as used herein, refers to effectual stoppage of action or progress.

The terms “reduce”, “decrease”, and “lower” are used synonymously herein, to refer to a diminishing, a decrease in, an attenuation or abatement of the degree, intensity, extent, size, amount, density or number of.

The term “retinopathy” as used herein refers to an eye disease that is caused by damage to the small blood vessels in the retina. Loss of vision may result.

The term “risk factor” as used herein refers to anything that raises the chances of a person developing a disease.

The term “side effects” as used herein refers to the unintended action(s) of a drug.

The terms “subject” and “patient” are used interchangeably herein to refer to animal species of mammalian origin that may benefit from the administration of a drug composition or method of the described invention. Examples of subjects include humans, and other animals such as horses, pigs, cattle, dogs, cats, rabbits, mice, rats and aquatic mammals.

The terms “therapeutic amount”, “therapeutically effective amount” and “amount effective” are used interchangeably herein to refer to an amount of one or more active agent(s) that is sufficient to provide the intended benefit of treatment. Dosage levels are based on a variety of factors, including the type of injury, the age, sex, weight, medical condition of the patient, the severity of the condition, the route of administration and the particular active agent employed. The dosage regimen may vary widely, but can be determined routinely by a physician using standard methods.

The term “treat”, “treating” or “to treat” as used herein, refers to accomplishing one or more of the following: (a) reducing the severity of a disorder; (b) limiting the development of symptoms characteristic of a disorder being treated; (c) limiting the worsening of symptoms characteristic of a disorder being treated; (d) limiting the recurrence of a disorder in patients that previously had the disorder; and (e) limiting recurrence of symptoms in patients that were previously asymptomatic for the disorder. The term “treat”, “treating” or “to treat” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms.

The term “triglyceride” as used herein refers to the storage form of fat in the body. High triglyceride levels may occur when diabetes is out of control.

The term “Type 1 diabetes” as used herein refers to a condition characterized by high blood glucose levels caused by a total lack of insulin that occurs when the body's immune system attacks the insulin-producing beta cells in the pancreas and destroys them. The pancreas then produces little or no insulin. Type 1 diabetes develops most often in young people but can appear in adults.

The term “Type 2 diabetes” as used herein refers to a condition characterized by high blood glucose levels caused by either a lack of insulin or the body's inability to use insulin efficiently. Type 2 diabetes develops most often in middle-aged and older adults but can appear in young people.

According to one aspect, the described invention provides a method for treating diabetes in a mammal comprising administering to the mammal a therapeutic amount of a glucagon depleting compound, wherein the therapeutic amount of the glucagon depleting compound is effective to elicit a bihormonal response in the mammal.

According to another aspect, the described invention provides a method for treating hyperglycemia in a mammal comprising administering to the mammal a therapeutic amount of a glucagon depleting compound, wherein the therapeutic amount of the glucagon depleting compound is effective to elicit a bihormonal response in the mammal.

According to another aspect, the described invention provides a method for eliciting a bihormonal response in a mammal suffering from diabetes comprising administering to the mammal a therapeutic amount of a glucagon depleting compound.

According to one embodiment, the bihormonal response is a normalization of glucagon levels and a normalization of insulin levels.

According to one embodiment, the therapeutic amount of the glucagon depleting compound is effective to normalize blood glucose levels in the mammal. According to another embodiment, the therapeutic amount of the glucagon depleting compound is effective to normalize glycated hemoglobin (HgAlC) levels in the mammal. According to another embodiment, the therapeutic amount of the glucagon depleting compound is effective to normalize glucagon levels in the mammal. According to another embodiment, the therapeutic amount of the glucagon depleting compound is effective to normalize C peptide levels in the mammal. According to another embodiment, the therapeutic amount of the glucagon depleting compound is effective to inhibit the expression of glucagon mRNA.

According to one embodiment, the diabetes is Type 1 diabetes. According to another embodiment, the diabetes is Type 2 diabetes.

According to one embodiment, the hyperglycemia is associated with Type 1 diabetes. According to another embodiment, the hyperglycemia is associated with Type 2 diabetes.

According to one embodiment, the described invention provides a method of treating or inhibiting the progression of type 1 diabetes in a mammal. The method includes the steps of administering a therapeutically-effective amount of a glucagon depleting compound of the described invention to a mammal suffering from type I diabetes. According to one embodiment, the glucagon depleting drug is compound A. According to another embodiment, the administration of the glucagon depleting compound elicits a bihormonal response. According to another embodiment, the bihormonal response is a normalization of glucagon levels and a normalization of insulin levels. According to another embodiment, the administration can be oral. According to another embodiment, the administration can be by injection or i.v. drip. According to another embodiment, the injection can be, for example, an intraperitoneal injection, a subcutaneous injection, or an intramuscular injection. According to another embodiment, the efficacy of treating or inhibiting the progression of type 1 diabetes in a mammal may be demonstrated by, for example, a normalization of four pathological characteristics of diabetes: blood glucose, glycated hemoglobin (HgAlC), glucagon and C peptide levels.

It is understood that the therapeutically-effective amount of the glucagon depleting compound will depend on factors including, but not limited to, the body weight of the mammal being treated and the stage of advancement of the disease. According to one embodiment, the therapeutically-effective amount may vary about a mean of about 0.2 mg/kg body weight.

According to one embodiment, the described invention provides a method of treating or inhibiting the progression of type 2 diabetes in a mammal. The method includes the steps of administering a therapeutically-effective amount of a glucagon depleting compound of the described invention to a mammal suffering from type 2 diabetes. According to another embodiment, the glucagon depleting compound is compound A. According to another embodiment, the administration of the glucagon depleting compound elicits a bihormonal response. According to another embodiment, the bihormonal response is a normalization of glucagon levels and a normalization of insulin levels. According to another embodiment, the administration can be oral. According to another embodiment, the administration can be by injection or i.v. drip. According to another embodiment, the injection can be, for example, an intraperitoneal injection, a subcutaneous injection, or an intramuscular injection. According to another embodiment, the efficacy of treating or inhibiting the progression of type 2 diabetes in a mammal may be demonstrated by, for example, a normalization of blood glucose levels with time of treatment. According to one embodiment, normalization of blood glucose levels is approximately 100 mg/dL.

The compositions of the present invention may be in a form suitable for oral use, for example, as tablets, troches, lozenges, aqueous or oily suspensions, dispersible powders or granules, emulsions, hard or soft capsules or syrups or elixirs. As used herein, the terms “oral” or “orally” refer to the introduction into the body by mouth whereby absorption occurs in one or more of the following areas of the body: the mouth, stomach, small intestine, lungs (also specifically referred to as inhalation), and the small blood vessels under the tongue (also specifically referred to as sublingually). Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be formulated for controlled release.

Compositions of the present invention also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the present invention may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the present invention may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the present invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents also may be present.

The compositions of the invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

The compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the present invention may take the form of tablets or lozenges formulated in a conventional manner.

The compositions of the present invention may be in the form of a sterile injectable aqueous or oleaginous suspension. The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or infusion techniques. A parenterally administered composition of the present invention is delivered using a needle, e.g., a surgical needle. The term “surgical needle” as used herein, refers to any needle adapted for delivery of fluid (i.e., capable of flow) compositions of the present invention into a selected anatomical structure. Injectable preparations, such as sterile injectable aqueous or oleaginous suspensions, may be formulated according to the known art using suitable dispersing or wetting agents and suspending agents.

The sterile injectable preparation may also be a sterile injectable solution or suspension in a nontoxic parenterally acceptable diluent or solvent, for example, as a solution in 1,3-butanediol. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances, which increase the viscosity of the suspension and include, for example, sodium carboxymethyl cellulose, sorbitol and/or dextran. Optionally, the suspension may also contain stabilizers.

The term “dispersion”, as used herein, refers to a two-phase system, in which one phase is distributed as particles or droplets in the second, or continuous phase. In these systems, the dispersed phase frequently is referred to as the discontinuous or internal phase, and the continuous phase is called the external phase or dispersion medium. For example, in coarse dispersions, the particle size is 0.5 mm. In colloidal dispersions, size of the dispersed particle is in the range of approximately 1 nm to 0.5 mm. Molecular dispersion is a dispersion, in which the dispersed phase consists of individual molecules; if the molecules are less than colloidal size, the result is a true solution.

The compositions of the present invention may be in the form of a dispersible dry powder for delivery by inhalation or insufflation (either through the mouth or through the nose). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the present invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container that provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

The compositions of the present invention may be in the form of suppositories for rectal administration of the composition. “Rectal” or “rectally” as used herein refers to introduction into the body through the rectum where absorption occurs through the walls of the rectum. These compositions can be prepared by mixing the drug with a suitable nonirritating excipient such as cocoa butter and polyethylene glycols, which are solid at ordinary temperatures but liquid at the rectal temperature and will therefore melt in the rectum and release the drug. When formulated as a suppository the compositions of the invention may be formulated with traditional binders and carriers, such as triglycerides.

The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. For the purpose of this application, topical applications shall include mouthwashes and gargles.

Topical administration may also involve the use of transdermal administration such as transdermal patches or iontophoresis devices, which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

According to some embodiments, the compositions of the present invention may be formulated with an excipient or carrier selected from solvents, suspending agents, binding agents, fillers, lubricants, disintegrants, and wetting agents/surfactants/solubilizing agents. The terms “excipient” or “carrier” refer to substances that do not deleteriously react with the glucagon-depleting compounds. Carriers must be of sufficiently high purity and of sufficiently low toxicity to render them suitable for administration to the subject being treated. The carrier can be inert, or it can possess pharmaceutical benefits.

The carrier can be liquid or solid and is selected with the planned manner of administration in mind to provide for the desired bulk, consistency, etc., when combined with an active and the other components of a given composition. Typical pharmaceutical carriers include, but are not limited to, binding agents (including, but not limited to pregelatinized maize starch, polyvinylpyrrolidone or hydroxypropyl methylcellulose); fillers (including but not limited to lactose and other sugars, microcrystalline cellulose, pectin, gelatin, calcium sulfate, ethyl cellulose, polyacrylates or calcium hydrogen phosphate.); lubricants (including, but not limited to magnesium stearate, talc, silica, sollidal silicon dioxide, stearic acid, metallic stearates, hydrogenated vegetable oils, corn starch, polyethylene glycols, sodium benzoate, sodium acetate); disintegrants (including, but not limited to, starch, sodium starch glycolate) and wetting agents (including but not limited to sodium lauryl sulfate). Additional suitable carriers for the compositions of the present invention include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like, which do not deleteriously react with the glucagon-depleting compounds.

The term “pharmaceutically acceptable carrier” as used herein refers to any substantially non-toxic carrier conventionally useful for administration of pharmaceuticals in which the glucagon-depleting compounds will remain stable and bioavailable. In some embodiments, the pharmaceutically acceptable carrier of the compositions of the described invention include a release agent such as a sustained release or delayed release carrier. In such embodiments, the carrier can be any material capable of sustained or delayed release of the glucagon-depleting lcompounds of the described invention to provide a more efficient administration, resulting in less frequent and/or decreased dosage of the active ingredient, ease of handling, and extended or delayed effects. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

In some embodiments, the compositions of the present invention can further include one or more compatible active ingredients in addition to the glucagon-depleting compounds of the described invention, which are aimed at providing the composition with another pharmaceutical effect in addition to that provided by the glucagon-depleting compounds of the described invention. “Compatible” as used herein means that the active ingredients of such a composition are capable of being combined with each other in such a manner so that there is no interaction that would substantially reduce the efficacy of each active ingredient or the composition under ordinary use conditions.

A composition of the present invention, alone or in combination with other active ingredients, may be administered to a subject in a single dose or multiple doses over a period of time. As used herein, the terms “therapeutically effective amount,” and “pharmaceutically effective amount” are used interchangeably to refer to the amount of the composition of the invention that results in a therapeutic or beneficial effect, including a subject's perception of health or general well-being, following its administration to a subject.

The concentration of the active substance is selected so as to exert its therapeutic effect, but low enough to avoid significant side effects within the scope and sound judgment of the skilled artisan. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors. Those of skill in the art can readily evaluate such factors and, based on this information, determine the particular effective concentration of a composition of the present invention to be used for an intended purpose.

A skilled artisan can determine a pharmaceutically effective amount of the inventive compositions by determining the unit dose. As used herein, a “unit dose” refers to the amount of inventive composition required to produce a response of 50% of maximal effect (i.e. ED50). The unit dose can be assessed by extrapolating from dose-response curves derived from in vitro or animal model test systems. The amount of compounds in the compositions of the present invention that will be effective in the treatment of a particular disorder or condition will depend on the nature of the disorder or condition, and can be determined by standard clinical techniques (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN'S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulation will also depend on the route of administration, and the seriousness of the disease or disorder, and should be decided according to the judgment of the practitioner and each patient's circumstances. Various administration patterns will be apparent to those skilled in the art.

The dosage ranges for the administration of the compositions of the present invention are those large enough to produce the desired therapeutic effect. Preferably, the therapeutically effective amount of the compositions of the present invention is administered one or more times per day on a regular basis.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural references unless the context clearly dictates otherwise. All technical and scientific terms used herein have the same meaning.

The publications discussed herein are provided solely for their disclosure prior to the filing date of the present application and each is incorporated by reference in its entirety. Nothing herein is to be construed as an admission that the described invention is not entitled to antedate such publication by virtue of prior invention. Further, the dates of publication provided may be different from the actual publication dates, which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Screening of Compound Library to Identify Compounds that Inhibit Glucagon Secretion

InR1G9 cells are glucagon secreting hamster islet cells (Takaki et al., 1986, In Vitro Cell. & Dev. Biol. 22:120) that exhibit characteristics of human islet α cells (e.g., response to insulin and known activators of glucagon secretion). Because InR1G9 cells share the functional characteristics of human islet α cells, InR1G9 cells are used as a reliable model for predicting human islet a cell responses in vivo.

InR1G9 cells were used to screen ˜200,000 compounds from the University of Texas Southwestern (UTSW) chemical library to identify compounds that inhibit glucagon secretion. This compound library encompasses ˜200,000 synthetic compounds that represent a large chemical space from several commercial vendors, including 1,200 marketed drugs from the Prestwick Chemical Library®, and 600 compounds that went to pre-clinical tests from the NIH library.

A cell-based method for compound screening was used. For example, InR1G9 cells were suspended in phenol red free DMEM media containing 25 mM glucose (Gibco, catalog number 31053-028)) and 1×10⁴ cells were dispensed into each well of a 384-well plate (PerkinElmer Cultureplate-384; Catalog Number 6007680) in a 30 uL volume. After two hour incubation at 37° C. to allow cells to attach to the plate, 0.3 uL of compound (5 uM, final concentration) in DMSO was added to the wells in a one compound per well format. Each compound was run as a singlet. Cells were grown at 37° C. for 20 hours. The plate was removed from the incubator and cooled to room temperature, then the four reagents were added individually using a Biotek MicroFill instrument (AF100A0. First, 5 μL of the anti-glucagon antibody (provided by Roger Unger) 1:10,000 dilution, final) was added to the wells for 30 minutes. Second, 5 μL of acceptor beads (PerkinElmer AlphaLISA Protein A Aceptor beads—250 ug, Catalog Number AL101C; 2 ug/ml, final) was added and incubated for 30 minutes. Third, 5 μL of biotinylated glucagon (AnaSpec, Biotin-Glucagon (1-29), bovine, human, procine, Catalog Number 60274-1; 1 nM, final) was added and allowed to incubate for an additional 60 minutes. Finally, 5 μL of donor beads (PerkinElmer Streptavidin Donor Beads—1 mg, Catalog Number 6760002S; 8 ug/ml, final) was added under green lighting (to eliminate red wavelengths) and allowed to incubate for 120 minutes before reading on an Envision plate reader (PerkinElmer). The total volume in each well was 50 uL. Competition between the glucagon present in the culture media and biotinylated glucagon lowered the AlphaLisa signal.

Compounds that inhibited glucagon secretion by >50% (half maximal inhibitory concentration or IC50) as compared to positive control wells (InR1G9 cells in the absence of compound), were identified. These compounds were selected as glucagon depleting compounds based on the following criteria: (i) they were not known to be toxic; (ii) they were not known to inhibit secretion of a different reporter molecule; (iii) they did not have undesirable chemical structures (e.g., metal chelators, chemically reactive compounds); and (iv) they had not been active in other screens of the UTSW compound library. From 900 such compounds, approximately 500 were tested for their ability to inhibit glucagon gene expression in InR1G9 cells treated with 5 μM of each compound overnight. Based on these criteria, eleven compounds were selected that inhibited glucagon gene expression, as measured by qPCR, by greater than 75%. From the eleven compounds, an exemplary compound, Compound A, was selected for further evaluation both in vitro and in vivo.

### Example 2

### Quantitative Reverse-Transcriptase Polymerase Chain Reaction (qRT-PCR) Analysis of Glucagon Gene Expression Inhibition by Compound A in InR1G9 Cells

Quantitative reverse-transcriptase polymerase chain reaction (qRT-PCR) was used to test whether Compound A could inhibit expression of the endogenous glucagon gene in InR1G9 cells.

For example, InR1G9 cells were cultured in Dulbecco's Modified Eagle's Medium (DMEM) (Life Technologies, Carlsbad, Calif., catalog number 1195-084) (4.5 g glucose/L) supplemented with 10% fetal bovine serum (Life Technologies, catalog number 10438-026), 1% penicillin (50 U/mL)/streptomycin (50 μg/mL) solution (Life Technologies, catalog number 15070-063) at 37° C. and 5% CO₂ in 96-well cell culture plates (Corning/Costar, Acton, Mass., catalog number CLS3595) at 1×10⁶ cells/well. Compound A was serially diluted 10-fold in dimethylsulfoxide (DMSO) (Sigma-Aldrich, St. Louis, Mo., catalog number 494429). A 50 μL aliquot of each dilution of Compound A was then added to separate wells of a 96-well cell culture plate containing 1×10⁶InR1G9 cells per well and incubated at 37° C. and 5% CO₂ for 18 hours. Following incubation, culture media was removed and total RNA was isolated from the cells usingRNeasy® Mini Kits according to the manufacturer's instructions (Qiagen Cat. No. 74106. Briefly, InR1G9 cells were pelleted by centrifugation (e.g., 12,000×g for 10 minutes at room temperature). The cell culture medium was aspirated; the pellet was resuspended in 350 μL of RLT Plus buffer and homogenized by vortexing for 30 seconds. Next, the homogenized lysate was transferred to a gDNA Eliminator spin column placed in a 2 mL collection tube and centrifuged for 30 seconds at ≧8,000×g. After centrifugation, the column was discarded and the flow-through was saved. Next, 350 μL of 70% ethanol was added to the flow-through and mixed by pipetting. The ethanol/flow-through mixture was transferred to an RNeasy® spin column placed in a 2 mL collection tube and centrifuged for 15 seconds at ≧8,000×g. After centrifugation, the flow-through was discarded, 700 μL of RW1 buffer was added to the RNeasy® spin column and the spin column was centrifuged for 15 seconds at ≧8,000×g. Again, flow-through was discarded, 500 μL of RPE buffer was added to the RNeasy® spin column and the spin column was centrifuged for 15 seconds at ≧8,000×g. Flow-through was discarded, 500 μL of RPE buffer was added to the RNeasy® spin column and the spin column was centrifuged for 2 minutes at ≧8,000×g. After centrifugation, the RNeasy® spin column was transferred to a new collection tube, 30 μL RNase-free water was added, and the spin column was centrifuged for 1 minute at ≧8,000×g to elute RNA.

Glucagon mRNA expression was measured from isolated total RNA using glucagon-specific primers and QuantiTect® SYBR® Green RT-PCR kit (Qiagen, Valencia, Calif., catalog number 204243) according to manufacturer's protocol. Briefly, 500 ng of isolated total RNA was added to PCR reaction tubes (Agilent Technologies/Stratagene, Santa Clara, Calif., catalog number Z376418-1PAK) containing 25 μL of 2× QuantiTect SYBR Green RT-PCR Master Mix, 0.5 μM glucagon-specific forward primer, 0.5 μM glucagon-specific reverse primer and 0.5 μL QuantiTect RT Mix. RNAse-free water was added to the PCR reaction tubes to bring the total volume of the reaction to 50 μL. 10 μL from each reaction mix was added to each of three wells of a 384-well reaction plate (ABI Prism 4309849, Applied Biosystems) PCR reaction tubes, which were placed in a thermocycler (e.g., 7900 HT from Applied Biosystems, Carlsbad, Calif.), programmed with the following cycling conditions:

Reverse Transcription: 30 minutes, 50° C.

PCR Initial Activation Step: 15 minutes, 95° C.

Denaturation: 15 seconds, 94° C.

Annealing: 30 seconds, 50-60° C.

Extension: 30 seconds, 72° C.

Number of Cycles: 35

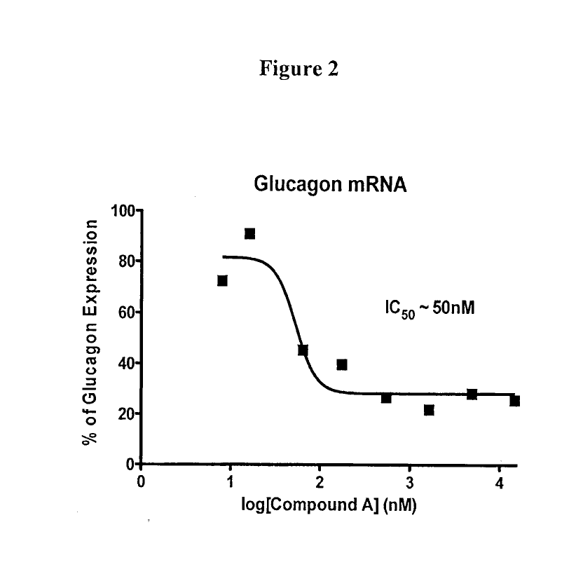

FIG. 2 shows the dose response curve for suppression of transcription of glucagon mRNA by Compound A in InR1G9 cells. Based on the dose response shown in FIG. 2, the half maximal inhibitory concentration (IC₅₀) for Compound A was calculated to be approximately 50 nM.

### Example 3

### Effect of Compound A on Blood Glucose in NOD Mice

Non-obese diabetic (NOD) mice are a well-established model system for human type 1 diabetes, since they are genetically well-characterized and spontaneously develop autoimmune T cell-mediated insulin-dependent diabetes, which shares many similarities to autoimmune or type 1 diabetes (T1D) in humans, such as the presence of pancreas-specific autoantibodies, autoreactive CD4+ and CD8+ T cells and genetic linkage to disease syntenic to that found in humans (Belizario J. E., The Open Immunology Journal, 2009, 2, 79-85).

NOD mice were used to test whether Compound A could lower blood glucose levels. Eight NOD mice were purchased from The Jackson Laboratory (Bar Harbor, Me.) and divided into two groups, experimental and control, consisting of 4 mice each. Mice in each group received the same diet and were housed under the same conditions. Mice in the experimental group were implanted with ALZET® osmotic pumps (Cupertino, Calif.) containing Compound A after the onset of autoimmune diabetes, while mice in the control group were implanted with ALZET® osmotic pumps containing saline after the onset of autoimmune diabetes. Mice in both the experimental and control groups were treated with Compound A and saline, respectively, for 75 days. The dose of Compound A delivered to mice in the experimental group was 0.2 mg/kg/day. Mice treated with Compound A ate normally (3 g/day) and maintained normal weight and activity and had normal urination (i.e., appeared healthy). The control mice died of complications of diabetes at or before 75 days.

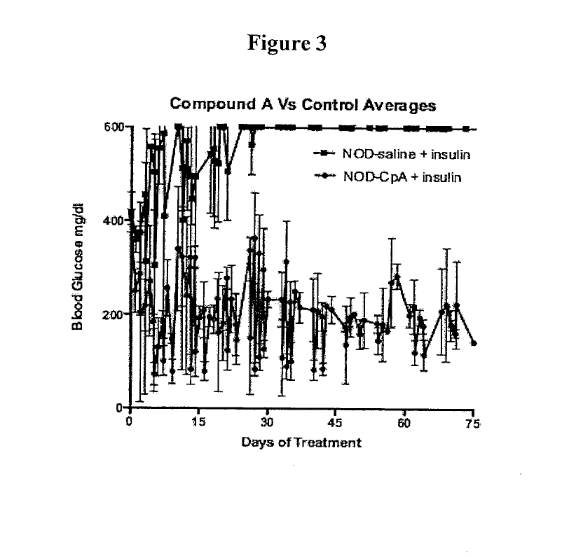

FIG. 3 shows the blood glucose levels measured in NOD mice treated with Compound A (circles) and NOD mice treated with saline (squares). Compound A was able to lower blood glucose levels in NOD mice overnight.

### Example 4

### Effect of Compound A on Triglycerides in NOD Mice

Insulin plays a central role in the regulation of lipid metabolism. (Vergès, Diabetes Metab., 27:223-227, 2001 and Bruno Vergès (2011), Lipid Disorders in Type 1 Diabetes, Type 1 Diabetes—Complications, Pathogenesis, and Alternative Treatments, Prof Chih-Pin Liu (Ed.), ISBN: 978-953-307-756-7, InTech, DOI: 10.5772/20869). It inhibits the production of Very Low Density Lipoprotein (VLDL), the precursor of Low Density Lipoprotein (LDL), from the liver. Insulin also reduces VLDL production by diminishing circulating free fatty acids, which are substrates for VLDL, and is a potent activator of lipoprotein lipase (LPL), thus promoting the catabolism of triglyceride-rich lipoproteins and consequently reducing plasma triglyceride levels. (Bruno Vergès (2011), Lipid Disorders in Type 1 Diabetes, Type 1 Diabetes—Complications, Pathogenesis, and Alternative Treatments, Prof Chih-Pin Liu (Ed.), ISBN: 978-953-307-756-7, InTech, DOI: 10.5772/20869). Moreover, insulin promotes the clearance of LDL by increasing LDL B/E receptor expression and activity (Chait et al., J. Clin. Invest., 64:1309-1319, 1979; Mazzone et al., Diabetes, 33:333-338, 1984; and Bruno Vergès (2011), Lipid Disorders in Type 1 Diabetes, Type 1 Diabetes—Complications, Pathogenesis, and Alternative Treatments, Prof Chih-Pin Liu (Ed.), ISBN: 978-953-307-756-7, InTech, DOI: 10.5772/20869). Likewise, insulin acts on High Density Lipoprotein (HDL) metabolism by activating Lecithin-Cholesterol Acyl Transferase (LCAT), which accounts for the synthesis of most of the cholesterol esters in plasma, as well as hepatic lipase activities (Ruotolo et al., Diabetes Care, 17:6-12, 1994 and Bruno Vergès (2011), Lipid Disorders in Type 1 Diabetes, Type 1 Diabetes—Complications, Pathogenesis, and Alternative Treatments, Prof. Chih-Pin Liu (Ed.), ISBN: 978-953-307-756-7, InTech, DOI: 10.5772/20869).

Patients suffering from insulin deficiency may exhibit a reduced LPL activity, which leads to profound reduction of triglyceride-rich lipoprotein catabolism resulting in hypertriglyceridemia (Taskinen, Diabetes Metab. Rev., 3:551-570, 1987 and Bruno Vergès (2011), Lipid Disorders in Type 1 Diabetes, Type 1 Diabetes—Complications, Pathogenesis, and Alternative Treatments, Prof. Chih-Pin Liu (Ed.), ISBN: 978-953-307-756-7, InTech, DOI: 10.5772/20869). Reduced catabolism of triglyceride-rich lipoproteins is, by far, the main factor involved in hypertriglyceridemia, which resolves rapidly after well-titrated insulin therapy (Weidman et al., J. Lipid Res., 23:171-182, 1982 and Bruno Vergès (2011), Lipid Disorders in Type 1 Diabetes, Type 1 Diabetes—Complications, Pathogenesis, and Alternative Treatments, Prof. Chih-Pin Liu (Ed.), ISBN: 978-953-307-756-7, InTech, DOI: 10.5772/20869).

The sphingolipid ceramide, a component of lipoproteins (Wiesner, P. et al., J. Lipid Res. 50: 574-585 (2009)) is elevated in the LDL of type 2 diabetic patients compared with insulin-sensitive individuals, independent of obesity. Boon, J. et al., Diabetes 62(2): 401-410 (2013). Weight loss reduces plasma ceramide.

Clinical data indicate that circulating ceramides correlate with systemic insulin resistance and inflammation (Haus, J M et al, Diabetes 58: 337-343 (2009). De Mello, V D et al, Diabetologia 52: 2612-2615 (2009)), and pharmacological inhibition of whole-body ceramide synthesis in obese mice decreases plasma ceramide, reduces inflammatory parameters, and improves insulin action (Holland, W L et al, Cell Metab. 5: 167-179 (2007); Yang, G et al, Am. J. Physiol. Endocrinol. Metab. 297: E211-E224 (2009)). Ceramide inhibits insulin signaling by several independent mechanisms: by increasing PP2A activity (Dobrowsky, R T et al, J. Biol. Chem. 268: 15523-15530 (1993)), which decreases Akt phosphorylation and activity (Resjö S, et al., Cell Signal 14:231-238 (2002)); by blocking the recruitment of Akt to the plasma membrane (Stratford, S. et al., J. Biol. Chem. 279: 26608-36615 (2004)), which is required for activation by the upstream kinases PDK1 (at Akt Thr308) and TORC2 (at Akt Ser473); and by ceramide accumulating in caveolin-enriched domains, activating PKCζ, which sequesters Akt in a repressed state within these membrane domains to prevent insulin signaling (Blouin, C M et al, Diabetes 59: 600-610 (2010)).

Non-obese diabetic (NOD) mice were used to test whether Compound A could lower triglyceride levels.

NOD mice were purchased from The Jackson Laboratory (Bar Harbor, Me.) and divided into two groups, experimental and control, consisting of 1 mouse each. A wild-type mouse (The Jackson Laboratory, Bar Harbor, Me.) was included in this study as an indicator of normal triglyceride levels. The mouse in each group received the same diet and was housed under the same conditions. The experimental mouse was implanted with an ALZET® osmotic pump (Cupertino, Calif.) containing Compound A after the onset of autoimmune diabetes. The control mouse was implanted with an ALZET® osmotic pump containing saline after the onset of autoimmune diabetes. Both the experimental mouse and the control mouse were treated with Compound A and saline respectively, for 10 days. Both mice were also treated with insulin at 1/10 the dose required for normal insulin monotherapy of NOD mice. The dose of Compound A delivered to the experimental mouse was 0.2 mg/kg/day. The mouse treated with Compound A ate normally (3 g/day) and maintained normal weight and activity and had normal urination (i.e., appeared healthy). The control mouse died at or before 75 days.

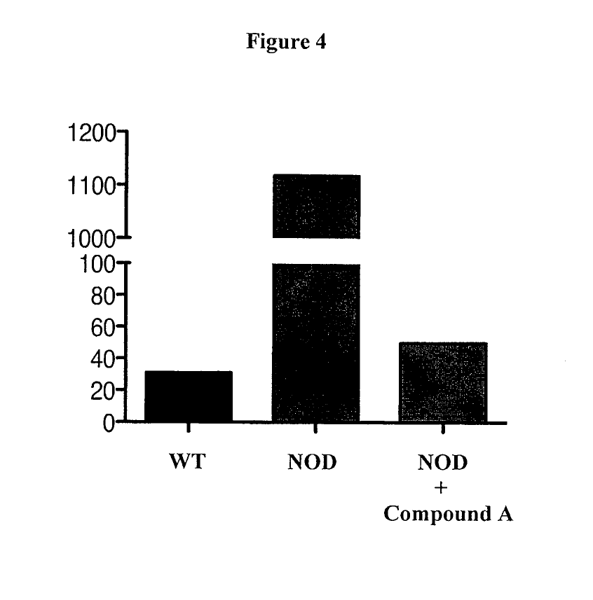

FIG. 4 shows triglyceride levels measured in the wild-type (wt) mouse, the NOD mouse treated with saline and the NOD mouse treated with Compound A. Compound A lowered blood triglyceride levels to near normal (as compared to wt).

### Example 5

### Effect of Compound A on Glycation of Proteins in NOD Mice

In this study, non-obese diabetic (NOD) mice were used to test whether Compound A could maintain a normal level of protein glycation.

A long-term effect of elevated blood glucose is the glycation of proteins. Rahbar et al. (Biochem. Biophys. Res. Commun. 36:838-843, 1969) first observed an increase in an “unusual” hemoglobin in patients with diabetes. It was later discovered that glucose binds red blood cells resulting in a “glycated” hemoglobin, or hemoglobin AlC (HgAlC) (Trivelli et al., N. Engl. J. Med. 284:353-357, 1971 and Bunn et al., Biochem. Biophys. Res. Commun. 67:103-109, 1975). Currently, HgAlC is widely used as a glycemic marker and is considered by the American Diabetes Association, in combination with blood glucose monitoring, to be the primary technique to assess glycemic control (American Diabetes Association, Executive Summary: Standards of Medical Care in Dabetes-2012, Diabetes Care 35 (Suppl. 1):S4-S10, 2012 and Alarcon-Casas Wright and Hirsch, Diabetes Spectrum, 25(3):141-148, 2012). HgAlC levels >6.5% are characteristic of poor glycemic control and are an early clinical indicator for a risk of developing complications related to diabetes, including: cardiovascular disease, kidney disease, ocular disease and the like.

Eight NOD mice were purchased from The Jackson Laboratory (Bar Harbor, Me.) and divided into two groups, experimental and control, consisting of 4 mice each. Four wild-type (wt) mice were also purchased from The Jackson Laboratory (Bar Harbor, Me.). Wild-type mice were included in this study as an indicator of normal HgAlC levels and for the purpose of data normalization. Mice in each group received the same diet and were housed under the same conditions. Mice in the experimental group were implanted with ALZET® osmotic pumps (Cupertino, Calif.) containing Compound A after the onset of autoimmune diabetes. Mice in the control group were implanted with ALZET® osmotic pumps containing saline after the onset of autoimmune diabetes. Mice in both the experimental and control groups were treated with Compound A and saline, respectively, for 45 days. The dose of Compound A delivered to mice in the experimental group was 0.2 mg/kg/day. Mice treated with Compound A ate normally (3 g/day) and maintained normal weight and activity and had normal urination (i.e., appeared healthy). Blood glucose, glycated hemoglobin (HgAlC), glucagon and connecting peptide (C-peptide) levels were measured at the beginning of treatment (New Onset Diabetic) and after 45 days of treatment. The control mice died at or before 75 days.

C-peptide bridges the insulin A and B chains in the proinsulin molecule. It is released in equimolar amounts together with insulin from pancreatic β cells as a byproduct of the enzymatic cleavage of proinsulin to insulin. C-peptide is considered to be a more reliable marker than insulin for the measurement of endogenous insulin production because, unlike insulin, C-peptide does not undergo hepatic clearance. Therefore, C-peptide has constant peripheral clearance at various plasma concentrations and in the presence of alterations in plasma glucose concentrations (Ludvigsson, Front Biosci (Elite Ed), 1(5):214-223, 2013 and Palmer et al., Diabetes, Vol. 53, pp. 250-264, 2004).

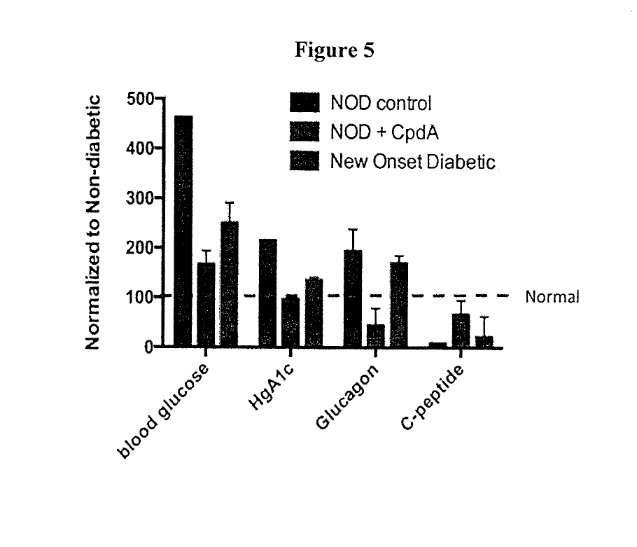

FIG. 5 shows that NOD mice treated with Compound A had normal levels of glycated hemoglobin (HgAlC) as compared to wild type. In addition, Compound A lowered glucagon levels below normal (as compared to wild type). Interestingly, mice treated with Compound A had elevated levels of C-peptide, indicating an increase in endogenous insulin production.

In summary, Compound A normalizes four pathological characteristics of diabetes: blood glucose, HgAlC, glucagon and C peptide.

### Example 6

### Effect of Compound A on Blood Glucose Levels of Mice with Diet-Induced Diabetes

Type 2 diabetes (T2D) is defined as a hyperglycemic disorder in which β-cells are present, thus distinguishing it from type 1 diabetes. Although numerous factors contribute to the development of T2D, the central defects are inadequate insulin secretion (insulin deficiency) and/or diminished tissue responses to insulin (insulin resistance) at one or more points in the complex pathways of hormone action (Triplitt, Am. J. Manag. Care 18 (1 Suppl) S4-S10, 2012). Insulin deficiency and insulin resistance frequently coexist, though the contribution to hyperglycemia can vary widely along the spectrum of T2D.

Histologically, α- and β-cells in patients with T2D do not appear to differ topographically from those of the nondiabetic pancreas (Unger and Orci, Proc. Natl. Acad. Sci., U.S., Vol. 107, No. 37, pp. 16009-16012, 2010). They are distributed in the same core-mantle arrangement in mice, and the frequency of contacts between α- and β-cells appears to be no less than in islets of non-diabetic humans. (Id.). The hyperglycemia of T2D is unaccompanied by the glycemic lability of T1D. (Id.). The stability of the hyperglycemia is attributed to restraint of glucagon secretion by β-cells juxtaposed to α-cells, thereby preventing both the absolute hyperglucagonemia of T1D and its extreme glycemic lability. (Id.).

T2D is characterized by “relative” hyperglucagonemia, meaning that the glucagon level is high relative to the ambient glucose level. (Id.) The α-cell dysfunction leading to hyperglucagonemia in T2D seems to result from failure of the juxtaposed β-cells to secrete the first phase of insulin, from insulin resistance of α-cells, or both. (Id.). A spike in insulin secretion may be the key paracrine signal for prompt glucagon suppression by glucose, since in its absence, glucagon is not suppressed and may even be stimulated by the increasing glucose. (Id.).

Many factors increase the risk of developing T2D, including family history, age, obesity, and lack of physical activity. However, the majority of patients with T2D are either obese (with obesity itself contributing to insulin resistance) or have an increased proportion of body fat in the abdominal region (Triplitt, Am. J. Manag. Care 18 (1 Suppl) S4-S10, 2012).

Diet-induced diabetic mice are a well-established model system for T2D. (Surwit et al., Diabetes 37:1163-1167, 1988). Diabetes in this model has been shown to be accompanied by two mechanistic characteristics for T2D: insulin resistance and islet dysfunction (Winzell and Ahren, Diabetes, Vol. 53, Supplement 3, pp. S215-S219, 2004).

Diet-induced diabetic mice were used to test whether Compound A could lower blood glucose levels. Two C57BL/6J mice were purchased from The Jackson Laboratory (Bar Harbor, Me.) and fed a high fat, high simple carbohydrate diet to induce diabetes as indicated by fasting blood glucose levels of >200 mg/dl. Both mice were implanted with an ALZET® osmotic pump (Cupertino, Calif.) containing Compound A after the onset of diabetes. Both mice were treated with Compound A for 30 days. The dose of Compound A delivered to mice was 0.2 mg/kg/day. Both mice ate normally (3 g/day) and maintained normal weight and activity and had normal urination (i.e., appeared healthy). Blood glucose levels were measured for 30 days prior to treatment with Compound A and for 30 days after commencing treating with Compound A.

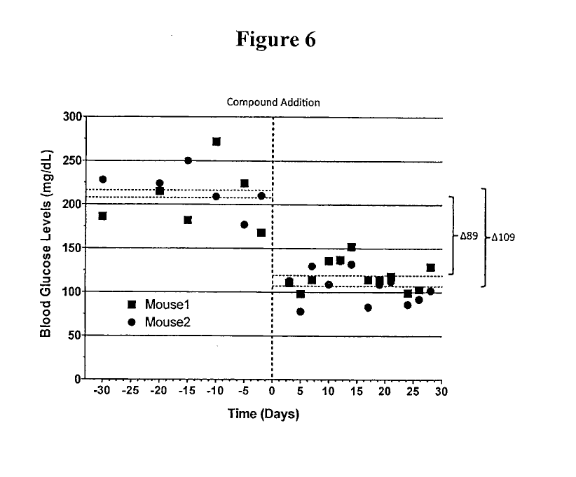

FIG. 6 shows that Compound A normalized (˜100 mg/dL) blood glucose in both diet-induced diabetic mice. This suggests that Compound A can be used to treat at least hyperglycemia in T2D.

While the present invention has been described with reference to the specific embodiments thereof, it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for treating diabetes in a mammal comprising administering to the mammal a therapeutic amount of a glucagon depleting compound, wherein the therapeutic amount of the glucagon depleting compound is effective to elicit a bihormonal response in the mammal.

2. The method according to claim 1, wherein the bihormonal response is a normalization of glucagon levels and a normalization of insulin levels.

3. The method according to claim 1, wherein the therapeutic amount of the glucagon depleting compound is effective to normalize blood glucose levels in the mammal.

4. The method according to claim 1, wherein the therapeutic amount of the glucagon depleting compound is effective to normalize glycated hemoglobin (HgAlC) levels and advanced glycation end-products in the mammal.

5. The method according to claim 1, wherein the therapeutic amount of the glucagon depleting compound is effective to normalize glucagon levels in the mammal.

6. The method according to claim 1, wherein the therapeutic amount of the glucagon depleting compound is effective to normalize C peptide levels in the mammal.

7. The method according to claim 1, wherein the therapeutic amount of the glucagon depleting compound is effective to inhibit the expression of glucagon mRNA.

8. The method according to claim 1, wherein the diabetes is Type 1 diabetes.

9. The method according to claim 1, wherein the diabetes is Type 2 diabetes.

10. A method for treating hyperglycemia in a mammal comprising administering to the mammal a therapeutic amount of a glucagon depleting compound, wherein the therapeutic amount of the glucagon depleting compound is effective to elicit a bihormonal response in the mammal.

11. The method according to claim 10, wherein the bihormonal response is a normalization of glucagon levels and a normalization of insulin levels.

12. The method according to claim 10, wherein the therapeutic amount of the glucagon depleting compound is effective to normalize blood glucose levels in the mammal.

13. The method according to claim 10, wherein the therapeutic amount of the glucagon depleting compound is effective to normalize glycated hemoglobin (HgAlC) levels and advanced glycation end-products in the mammal.

14. The method according to claim 10, wherein the therapeutic amount of the glucagon depleting compound is effective to normalize glucagon levels in the mammal.

15. The method according to claim 10, wherein the therapeutic amount of the glucagon depleting compound is effective to normalize C peptide levels in the mammal.

16. The method according to claim 10, wherein the therapeutic amount of the glucagon depleting compound is effective to inhibit the expression of glucagon mRNA.

17. The method according to claim 10, wherein the hyperglycemia is associated with Type 1 diabetes.

18. The method according to claim 10, wherein the hyperglycemia is associated with Type 2 diabetes.

19. A method for eliciting a bihormonal response in a mammal suffering from diabetes comprising administering to the mammal a therapeutic amount of a glucagon depleting compound.

20. The method according to claim 19, wherein the bihormal response is a normalization of glucagon levels and a normalization of insulin levels.

21. The method according to claim 19, wherein the therapeutic amount of the glucagon depleting compound is effective to normalize blood glucose levels in the mammal.

22. The method according to claim 19, wherein the therapeutic amount of the glucagon depleting compound is effective to normalize glycated hemoglobin (HgAlC) levels and advanced glycation end-products in the mammal.

23. The method according to claim 19, wherein the therapeutic amount of the glucagon depleting compound is effective to normalize glucagon levels in the mammal.

24. The method according to claim 19, wherein the therapeutic amount of the glucagon depleting compound is effective to normalize C peptide levels in the mammal.

25. The method according to claim 19, wherein the therapeutic amount of the glucagon depleting compound is effective to inhibit the expression of glucagon mRNA.

26. The method according to claim 19, wherein the diabetes is a Type 1 diabetes.

27. The method according to claim 19, wherein the diabetes is a Type 2 diabetes.
